SEQUENCE LISTING (33 sequences)

SEQ ID NO: 1 - protein, 12 aa, human
KALIHLSDLREY

SEQ ID NO: 2 - protein, 19 aa, human
NPLFKSATTTVMNPKFAES

SEQ ID NO: 3 - protein, 10 aa, human
NPLFKSATTT

SEQ ID NO: 4 - protein, 9 aa, human
VMNPKFAES

SEQ ID NO: 5 - protein, 6 aa, human
NPLFKS

SEQ ID NO: 6 - DNA, 6 bp, human
ksattt

SEQ ID NO: 7 - protein, 4 aa, human
NPKF

SEQ ID NO: 8 - protein, 4 aa, human
NPLF

SEQ ID NO: 9 - protein, 4 aa, human
  misc_feature (3)..(3): Xaa can be any naturally occurring amino acid
NPXF

SEQ ID NO: 10 - protein, 9 aa, human immunodeficiency virus
RKKRRQRRR

SEQ ID NO: 11 - protein, 21 aa, Bufo bufo gargarizans
TRSSRAGLQFPVGRVHRLLRK

SEQ ID NO: 12 - protein, 26 aa, artificial
  A 27 amino acid peptide with a sequence based on the N-terminal fragment residues 1-12 of the neuropeptide glanin fused with the sequence of the wasp venom mastoparan (14 amino acids) via a linking lysine residue
GWTLNSAGYLLGKINKALAALAKKIL

SEQ ID NO: 13 - protein, 18 aa, artificial
  A model peptide (model amphipathic peptide)
KLALKLALKALKAALKLA

SEQ ID NO: 14 - protein, 16 aa, human
AAVALLPAVLLALLAP

SEQ ID NO: 15 - protein, 10 aa, human
VPMLKPMLKE

SEQ ID NO: 16 - protein, 28 aa, prion
MANLGYWLLALFVTMWTDVGLCKKRPKP

SEQ ID NO: 17 - protein, 18 aa, Murine
LLIILRRRIRKQAHAHSK

SEQ ID NO: 18 - protein, 21 aa, artificial
  Pep-1 consists of 3 domains 1) a hydrophobic typtophan-rich motif 2) a hydrophilic lysine-rich domain from NLS of simian virus 40 large T antigen; and, 3) a spacer domain (SQP)
KETWWETWWTEWSQPKKKRKV

SEQ ID NO: 19 - protein, 18 aa, porcine
RGGRLSYSRRRFSTSTGR

SEQ ID NO: 20 - protein, 15 aa, artificial
  peptide translocating motif - origin, a peptide library
SDLWEMMMVSLACQY

SEQ ID NO: 21 - protein, 12 aa, artificial
  peptide translocating motif - origin, a peptide library
TSPLNIHNGQKL

SEQ ID NO: 22 - protein, 16 aa, drosophila
RQIKIWFQNRRMKWKK

SEQ ID NO: 23 - DNA, 12 bp, human
aatccccttttc

SEQ ID NO: 24 - DNA, 18 bp, human
aagagcgccaccacgacg

SEQ ID NO: 25 - DNA, 12 bp, human
aaccccaagttt

SEQ ID NO: 26 - protein, 6 aa, human
KSAVTT

SEQ ID NO: 27 - protein, 7 aa, human
KSAITTT

SEQ ID NO: 28 - protein, 4 aa, human
NPLY

SEQ ID NO: 29 - protein, 4 aa, human
NPRF

SEQ ID NO: 30 - protein, 16 aa, human
YRRFEKEKLKSQWNND

SEQ ID NO: 31 - protein, 4 aa, human
  misc_feature: X can be any naturally occuring amino acid
NPXY

SEQ ID NO: 32 - protein, 9 aa, artificial
  peptide translocating motif - consists of a polymer of arginine
RRRRRRRRR

SEQ ID NO: 33 - protein, 46 aa, Human
KALIHLSDLREYRRFEKEKLKSQWNNDNPLFKSATTTVMNPKFAES

(19) United States
(12) Patent Application Publication
(10) Pub. No.: US 2009/0118174 A1
(43) Pub. Date: May 7, 2009

(54) Novel peptides and methods for the treatment of inflammatory disorders

(75) Inventors: Geoffrey Wayne Krissansen, Auckland (NZ); Rupinder Kaur Kanwar, Auckland (NZ)

Correspondence Address: OCCHIUTI ROHLICEK & TSAO, LLP, 10 FAWCETT STREET, CAMBRIDGE, MA, 02138, US

(73) Assignee: Auckland Uniservices Limited, Auckland (NZ)

(21) Appl. No.: 11/911,980

(22) PCT Filed: Apr. 19, 2006
(86) PCT No.: PCT/NZ06/00080

§ 371 Date: May 23, 2008

(30) Foreign Application Priority Data
Apr. 19, 2005 (AU) 2005901975

(51) Int. Cl. A61K 38/17; C07K 5/00; C07K 7/00; C07K 14/47; A61K 38/08; G01N 33/566; A61K 31/711; A61P 29/00; C07K 16/18; A61K 38/10; C12N 15/12; C12N 15/85; A61K 38/07

(52) U.S. Cl. 514/12; 530/330; 530/329; 530/326; 530/328; 530/324; 536/23.5; 435/320.1; 514/18; 514/17; 514/13; 514/15; 436/501; 530/387.9; 435/7.24; 514/44

(57) ABSTRACT

Novel peptides, nucleic acids encoding them, and derivatives of the peptides are described. The peptides and nucleic acids are of use in modulating β2 integrin function and in treating β2 integrin-mediated inflammatory disorders.

### FIELD

The present invention relates to novel peptides, nucleic acids encoding same, pharmaceutical compositions comprising said peptides or nucleic acids, methods for modulating β2 integrin function, including methods for the treatment of inflammatory disorders, antibodies directed to said peptides, and methods for the identification of integrin β2 functional interactors.

### BACKGROUND

The precise control of leukocyte adhesivity is critical in maintaining effective homeostasis of the immune response, for lymphocyte motility, homing, and recirculation, the localization of leukocytes at sites of inflammation, and antigen presentation. A small subset of integrins, namely α4, β2, and β7 integrins, largely controls leukocyte adhesion, and related functions (1).

The integrins are a superfamily of transmembrane receptors which mediate cell-extracellular matrix and cell-cell interactions. Each integrin consists of noncovalently paired alpha and beta subunits. There are presently 8 beta and 18 alpha subunits known. The β2 subunit partners with alpha subunits to form the heterodimeric molecules, αLβ2, αMβ2, αXβ2, and αDβ2.

Integrin adhesivity is regulated by a complex array of intracellular signalling pathways that impinge on integrin subunit cytoplasmic domains, and trigger changes in integrin conformation, clustering (2), affinity for ligands (3, 4), and cell spreading (5), all of which contribute to increased cell adhesion (6-8).

αLβ2 mediates the adhesion of leukocytes to the intercellular adhesion molecules ICAM-1, ICAM-2, and ICAM-3, which are inducibly or constitutively expressed on many cell types, whereas αMβ2 and αXβ2 interact with an assortment of ligands including ICAM- and serum factors. αDβ2, which binds preferentially to ICAM-3, is strongly expressed on tissue-compartmentalized cells such as macrophage foam cells found in aortic fatty streaks that may develop into atherosclerotic lesions.

The β2 integrins are expressed on blood leukocytes in an inactive state necessary to maintain haemostasis. They are transiently activated by inflammatory cytokines, chemokines, divalent cations, and other agonists by both “outside-in” and “inside-out signalling” pathways that impinge on their cytoplasmic domains, and potentiate the adhesive functions of the extracellular domains (reviewed in 9 and 10). Mn++ is a known powerful activator of integrins. Mn⁺⁺ induces the clustering of integrins by a process that is blocked by inhibitors of intracellular kinases suggesting that the extracellular and intracellular domains of integrins are functionally linked (Dormond O, Ponsonnet L, Hasmim M, Foletti A, Ruegg C. Manganese-induced integrin affinity maturation promotes recruitment of alpha V beta 3 integrin to focal adhesions in endothelial cells: evidence for a role of phosphatidylinositol 3-kinase and Src. Thromb Haemost. 2004; 92(1):151-61. Depending on the cellular context the process of “inside-out signalling” is either positively or negatively regulated by several pathways involving protein kinase C, calcium/calmodulin kinase II (CaMKII), small GTP-binding proteins (Rac, Rho, Rnd1, R-ras, and H-ras), phosphatidylinositol 3-kinase, and unidentified protein tyrosine kinases. Such pathways can increase cell binding by causing changes in the affinity of an integrin for its ligand, induce integrin clustering, cell spreading, and/or modify the membrane and cytoskeleton to make it more pro-adhesive (11). The mechanisms by which the small integrin cytoplasmic domains transmit signals that alter the function of the extracellular domain is not known.

In resting leukocytes, β2 integrins are constitutively linked to the actin cytoskeleton via talin, where activation of cells induces transient proteolysis and dissociation of talin followed by reattachment of actin filaments to integrins mediated by the protein α-actinin, which may promote firm adhesion (12). Regulated binding of talin to integrin D tails is a final common element of cellular signaling cascades that control integrin activation (13). Binding of the talin head domain to the β2 subunit has been shown to activate αLβ2 concomitant with spatial separation of the αL and β2 cytoplasmic domains (14). Other proteins that interact directly with integrin cytoplasmic domains probably also alter integrin function (15). The β2 subunit cytoplasmic domain also interacts with the cytoskeletal protein filamin, Rack1, and cytohesin-1 which induces beta 2 integrin-dependent T cell adhesion (16). RanBPM interacts with the cytoplasmic domain of the β2 subunit, and synergizes with LFA-1-mediated adhesion in the transcriptional activation of an AP-1-dependent promoter (17).

Notwithstanding the above, the regulatory sites or motifs present within the integrin subunits have not been fully characterised. Accordingly, there is still much to be understood of the precise mechanisms which allow for regulation of integrin activity and concomitantly cell-cell or cell-extracellular matrix interactions.

In light of the role β2 integrins play in regulating leukocyte activity and targeting, and their implication in the development of certain inflammatory disorders, elucidating the precise mechanisms by which their function may be regulated may allow for control thereof, with concomitant amelioration of relevant inflammatory disorders.

Bibliographic details of the publications referred to herein are collected at the end of the description.

### OBJECT

It is an object of the present invention to provide novel peptides, nucleic acids encoding same, derivatives of said peptides, pharmaceutical compositions comprising said peptides, derivatives thereof, or nucleic acids, methods for modulating β2 integrin function, including methods for the treatment of inflammatory disorders, antibodies directed to said peptides, and/or methods for the identification of integrin β2 functional interactors and interference molecules and use of the peptides in designing mimetics thereof.

It is a further, or alternative object to at least provide the public with a useful choice of any one or the above.

### STATEMENT OF INVENTION

In accordance with the present invention the inventors have identified functional motifs in the β2 cytoplasmic domain that control the adhesion of β2 integrins. It has been surprisingly discovered that these functional motifs (sequences), which map within a region from residues 751-769 of the cytoplasmic tail of the β2 subunit, provide peptides which in isolation as free peptides inhibit the adhesion of β2 integrins to their ligands, as is exemplified hereinafter in relation to αLβ2-mediated adhesion of human H9 T cells, to ICAM-1. Peptides carrying the motifs, or nucleic acids encoding same, may provide novel anti-inflammatory reagents for the treatment of inflammatory disorders.

Accordingly, in one aspect of the present invention there is provided an isolated peptide comprising at least the amino acid sequence of any one of:

NPxF
KSATTT

or a derivative of said peptide,
wherein x is any amino acid.

Preferably x is K or L.

In another aspect, the present invention provides a peptide consisting of the amino acid sequence of any one of:

NPLFKSATTTVMNPKFAES
NPLFKSATTT
VMNPKFAES
NPLFKS

or a derivative of said peptide.

In another aspect, the invention provides a peptide consisting of the amino acid sequence KALIHLSDLREYRRFEKEKLKSQWNNDNPLFKSATTTVMNPKFAES, or a derivative thereof.

In a related aspect, the invention provides a peptide as herein before described, or a derivative thereof, together with a cell membrane translocating motif. The motif may be fused with, conjugated to, or otherwise incorporated in the peptide. Preferably, said cell membrane translocating motif is peptide-based. More preferably, said cell membrane translocating motif is penetratin or a polymer of arginine.

In another aspect, the present invention provides isolated nucleic acids which encode a peptide or a derivative thereof in accordance with the invention.

In a related aspect, the invention provides constructs or vectors comprising nucleic acids which encode a peptide or derivative thereof in accordance with the invention.

In another aspect, the invention provides an agent comprising at least a peptide, derivative thereof, or nucleic acid in accordance with the invention.

In a further aspect, the present invention provides a pharmaceutical composition comprising a peptide or derivative thereof in accordance with the invention, together with one or more pharmaceutically acceptable diluents, carriers and/or excipients.

In a related aspect, the present invention provides a pharmaceutical composition comprising a nucleic acid or construct in accordance with the invention together with one or more pharmaceutically acceptable diluents, carriers and/or excipients.

In a further aspect of the present invention there is provided a method for modulating the function of integrin β2 in a subject comprising at least the step of administering to said subject an effective amount of at least a peptide, or a derivative thereof as herein before described. The peptide or derivative thereof may be administered in the form of a composition as herein before described.

Alternatively, the method of modulating the function of integrin β2 in a subject comprises at least the step of administering to said subject an effective amount of at least a nucleic acid or construct as herein before described. The nucleic acid or construct may be administered in the form of a composition as herein before described.

In a further aspect, the present invention provides a method of modulating the function of integrin β2 in an in vitro system the method comprising at least the step of administering to said system a peptide, or a derivative thereof, nucleic acid, construct, or composition in accordance with the invention.

In a further aspect of the invention there is provided a method for the treatment of integrin β2-mediated inflammatory disorders comprising at least the step of administering to a subject in need thereof a therapeutically effective amount of at least a peptide, or a derivative thereof as herein before described. The peptide or derivative thereof may be administered in the form of a composition as herein before described.

In a further aspect of the invention there is provided a method for the treatment of integrin β2-mediated inflammatory disorders comprising at least the step of administering to a subject in need thereof a therapeutically effective amount of at least a nucleic acid or construct comprising same as herein before described. The nucleic acid or construct may be administered in the form of a composition as herein before described.

In another aspect, the present invention provides the use of a peptide, or a derivative thereof, nucleic acid, or construct as herein before described in the manufacture of a medicament for the treatment of integrin β2-mediated inflammatory disorders.

In yet a further aspect, the present invention provides a method for the identification of potential β2 integrin functional interactors (including interference molecules), of the peptides of the invention, the method comprising at least the step of bringing a potential functional interactor in contact with a peptide of the invention, or a derivative thereof, and observing whether or not binding occurs.

In a related aspect of the invention the method further comprises the step of determining whether or not the functional interactor molecule influences the level of adhesion of leukocytes to β2 integrin ligands. Preferably the method comprises the step of determining whether or not the functional interactor molecule lowers the level of, or disrupts or prevents, adhesion of leukocytes to β2 integrin ligands.

In another aspect, the invention provides the use of a peptide or derivative thereof in accordance with the invention in identifying or screening for potential β2 integrin functional interactor molecules.

In a related aspect, the invention provides the use of a peptide or derivative thereof in accordance with the invention in designing mimetics of said peptide or derivative.

In another aspect, the invention provides an antibody directed against a peptide or derivative of the invention.

In another aspect, the invention provides nucleic acid aptamers of a peptide or derivative of the invention.

In another aspect, the invention provides a kit for modulating the function of integrin β2 or for the treatment of integrin β2-mediated inflammatory disorders, the kit comprising at least a peptide or derivative thereof in accordance with the invention.

In a related aspect, the invention provides a kit for modulating the function of integrin β2 or for the treatment of integrin β2-mediated inflammatory disorders, the kit comprising a nucleic acid or construct in accordance with the invention.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1 illustrate the cell permeable β2cyt peptide sequences, and their uptake into cells. (A) Peptides representing different regions of β2cyt used in the study. Peptides were either fused to penetratin (Pen) or a D-isomeric form of a nine amino acid arginine polymer (r9). The N-terminal K residue in the penetratin sequence was used to complete the KALIHLSDLREY (Seq ID No. 1) sequence at the N-terminus of β2cyt. Upper and lower case denote L- and D-enantiomers, respectively. (B) Peptide taken up into cells was detected by staining fixed and permeabilized cytospins with FITC-streptavidin. Representative images of β2cyt peptides taken up into H9 cells were visualized by fluorescence microscopy. Left-hand panels show nuclei stained with DAPI.

FIG. 2 illustrates three cell-permeable peptides from the C-terminal region of β2cyt inhibit the adhesion of T cells to ICAM-1. H9 cells were preincubated with increasing concentrations of the indicated peptides, activated with Mn²⁺, and added to wells coated with ICAM-1-Fc. Unlabeled adherent cells were counted (A), and the fluorescein counts of CMDF-labeled adherent cells was recorded (B, C). The parental peptide NPLFKSATTTVMNPKFAES (A) (Seq ID No. 2) was found to be active and was divided into two to give the active peptides NPLFKSATTT (Seq ID No. 3) and VMNPKFAES (Seq ID No. 4) (B), and then peptide NPLFKSATTT was in turn divided into two to give overlapping peptides NPLFKS (Seq ID No. 5) and KSATTT (Seq ID No. 6) (C). The core talin-binding motif NPKF (Seq ID No. 7) was also tested (C). Data are reported as mean±SD of two independent experiments performed in duplicate.

FIG. 3 provides a schematic comparison of the position of CARDs in integrin subunits. (A) The sequences of the β2, β3, and β7 subunits are aligned, and the positions of the CARDs are highlighted. Peptides found to be bioactive when isolated as stand-alone peptides are in bold, and binding sites of intracellular ligands are indicated. (B) Alignment of integin β subunits for comparison of CARD motifs. The divergent β4 and β8 subunits were omitted. The positions of the CARDs are highlighted.

### PREFERRED EMBODIMENT(S)

The following is a description of the present invention, including preferred embodiments thereof, given in general terms. The invention is further elucidated from the disclosure given under the section entitled “Examples” herein after, which provides experimental support for, and specific examples of, the invention.

The inventors of the present invention have identified regulatory motifs within the β2 integrin (GenBank accession number M15395) subunit. These motifs map to a region of the cytoplasmic tail of the β2 subunit from residues 751 to 769. While not wishing to be bound by any particular theory, the inventors propose that these motifs constitute cell adhesion regulatory domains (CARDs) that modulate the interaction of β2 expressing leukocytes with their extracellular matrix, and with endothelial and epithelial cells, dendritic cells and other cells expressing appropriate ligands.

The inventors have surprisingly found that peptides comprising at least the motif NPKF (Seq ID No. 7), or NPLF (Seq ID No. 8), or KSATTT (Seq ID No. 6) are able to disrupt the interaction of β2 integrins with their ligands, for example ICAM-1. The inventors have also identified that peptides having the sequences NPLFKS (Seq ID No. 5), NPLFKSATTT (Seq ID No. 3), VMNPKFAES (Seq ID No. 4) and NPLFKSATTTVMNPKFAES (Seq ID No. 2) similarly have this ability. The inventors contemplate that peptides having the core consensus sequence NPxF (where x is any amino acid residue) will have such activity. Whilst not wishing to be bound by any particular theory, the implication is that peptides of the invention compete for intracellular proteins that are critical in controlling the function of β2 integrins thereby modulating their cellular adhesion function.

On the basis of the above findings, a peptide of the invention may be used to modulate the cellular adhesion function of β2 integrins, particularly the adhesion of leukocytes to each other, to the extracellular matrix and to epithelial and endothelial cells, both in in vitro systems and in vivo. Such modulation has application in controlling β2 integrin-mediated inflammatory events, and particularly in the treatment of β2 integrin-mediated inflammatory disorders.

Similarly, the inventors contemplate the use of nucleic acids encoding peptides of the invention, and constructs or vectors comprising such nucleic acids, in methods for modulating the cellular adhesion function of β2 integrins, likewise including treatment of β2 integin mediated inflammatory disorders.

Peptides, their derivatives, nucleic acids encoding same, and constructs or vectors comprising said nucleic acids may be referred to herein as “agents” or “agents of the invention”. Such agents may solely comprise a peptide, its derivative, a nucleic acid encoding same, or a construct or vector comprising said nucleic acid. Alternatively, said agents may comprise a peptide, its derivative, a nucleic acid encoding same, or a construct or vector comprising said nucleic acid in conjunction with additional elements. By way of example, an agent comprising a nucleic acid vector encoding peptides of the invention may be a naked DNA or DNA packaged in an appropriate viral capsid.

Additionally, a peptide of the invention may be used in assays for the identification of β2 integrin functional interactor molecules which may bind to and/or modulate the function of β2 integrins. As used herein the terms “β2 integrin functional interactors” or “β2 integrin functional interactor molecules” and the like should be taken in their broadest context. They are intended to include those molecules which decrease activity or function of β2 integrins, as well as those that increase such activity or function. Such interactors include intracellular signalling molecules and other cellular components which may modulate the cellular adhesion function of the β2 integrins, and also potential therapeutic agents which may have application in treatment of disorders mediated by this function.

Furthermore, peptides of the invention may be used in assays to identify interference molecules directed against the cytoplasmic domain of the integrin β2 subunit. As used herein, “interference molecules” are those molecules which are adapted to bind to a region of the cytoplasmic domain of the integrin subunit including a peptide motif of the invention. Preferably such “interference molecules” block the interaction of at least a region of the cytoplasmic domain with other molecules, and more preferably block the function of the cytoplasmic domain of the integrin subunit. “Interference molecules” include, but are not limited to, antibodies and nucleic acid aptamers (for example, RNA and DNA aptamers).

“Interference molecules” may find use in modulating or inhibiting the activity and function of β2 integrins, including disrupting or preventing the interaction of β2 integrins with their ligands, for example ICAM-1, thus modulating the cellular adhesion function of the β2 integrins, and having application in controlling β2 integrin-mediated inflammatory events, and particularly in the treatment of β2 integrin-mediated inflammatory disorders.

Peptides of the invention may also be used to design mimetics of the peptides, including small molecule mimetics, which may be of use therapeutically.

The phrases “modulate adhesion of leukocytes to each other and to epithelial and endothelial cells”, “modulating the cellular adhesion function of β₂ integrins” or “regulate the function of β2 integrins”, and the like, are generally used herein to refer to down-regulation of function. However, the inventors contemplate situations where up-regulation of function of the β2 integrins may occur through use of peptides, nucleic acids, or constructs of the invention; for example, where the peptides competitively bind to functional interactors which may have a negative effect on β2 integrin function. Accordingly, up-regulation of the function of the β2 integrins is also encompassed by the present invention. To this end, while pharmaceutical compositions and methods are described herein after in relation to the treatment of inflammatory disorders, which implies down-regulation of β2 integrin function, it should be understood that they may equally be applicable to treatments where up-regulation of β2 integrin function is desirable.

The term “inflammatory disorder(s)” should be taken to mean any undesired physiological condition which involves inflammation, aberrant or otherwise. “Inflammation” should be broadly taken to mean a characteristic reaction of tissues to injury or disease, or foreign particles and noxious stimuli, resulting in one or more of redness, swelling, heat, pain and loss of function. In accordance with the present invention, such inflammatory disorders will be mediated by the action of β2 integrins, and include, but are not limited to, demyelinating diseases such as multiple sclerosis, Type I diabetes mellitus, inflammatory bowel disease, asthma, dermatitis, arthritis, gastritis, mucositis, graft-versus-host disease, hepatitis, psoriasis, Graves disease, septic shock, hemorrhagic shock, ischemia-reperfusion injury, arterial/vascular injury, transplant rejection and inflammation that impedes tissue/skin healing.

As used herein, the term “treatment” is to be considered in its broadest context. The term does not necessarily imply that subject is treated until total recovery.

Accordingly, “treatment” broadly includes the modulation or control of inflammation, or other β2 integrin-mediated event, aberrant or otherwise, amelioration of the symptoms or severity of a particular disorder, or preventing or otherwise reducing the risk of developing a particular disorder.

It will be appreciated by those of general skill in the art to which the invention relates that the present invention is applicable to a variety of different animals. Accordingly, a “subject” includes any animal of interest. In particular the invention is applicable to mammals, more particularly humans.

It should be understood that a peptide or protein in accordance with the invention, is an “isolated” or “purified” peptide or protein. An “isolated” or “purified” peptide or protein is one which has been identified and separated from the environment in which it naturally resides. It should be appreciated that ‘isolated’ does not reflect the extent to which the peptide has been purified or separated from the environment in which it naturally resides. Peptides of use in the invention may be purified from natural sources or derived by chemical synthesis or recombinant techniques.

It should be understood that a nucleic acid in accordance with the invention, is an “isolated” or “purified” nucleic acid. An “isolated” or “purified” nucleic is one which has been identified and separated from the environment in which it naturally resides. It should be appreciated that ‘isolated’ does not reflect the extent to which the nucleic has been purified or separated from the environment in which it naturally resides. Nucleic acids of use in accordance with the invention may be purified from natural sources, or preferably derived by chemical synthesis or recombinant techniques.

### Peptides

A peptide in accordance with the invention comprises at least the amino acid sequence NPxF (Seq ID No. 9) (preferably NPKF (Seq ID No. 7) or NPLF (Seq ID No. 8)), or KSATTT (Seq ID No. 6). While the peptide may consist solely of one of these motifs, it should be appreciated that larger peptides in which one or more of these motifs is incorporated are also encompassed by the present invention. In one preferred embodiment, the core motifs NPxF (Seq ID No. 9) (preferably NPKF (Seq ID No. 7) or NPLF (Seq ID No. 8)), or KSATTT (Seq ID No. 6) are extended at either or both of their N- or C-termini to include the full cytoplasmic domain of a O₂ subunit (for example, the peptide may consist the amino acid sequence KALIHLSDLREYRRFEKEKLKSQWNNDNPLFKSATTTVMNPKFAES (Seq ID No. 33) of human β2). In other preferred embodiments, the core motifs NPxF (Seq ID No. 9) (preferably NPKF (Seq ID No. 7) or NPLF (Seq ID No. 8)), or KSATTT (Seq ID No. 6) are extended at either or both of their N- or C-termini by an additional 1 to 30 amino acids taken from the cytoplasmic domain of a native β₂ subunit amino acid sequence (preferably human β2), more preferably 1 to 25 amino acids, 1 to 20 amino acids, 1 to 15 amino acids or 1 to 10 amino acids, and most preferably 1 to 6 amino acids. Accordingly, the peptides NPLFKS (Seq ID No. 5), NPLFKSATTT (Seq ID No. 3), VMNPKFAES (Seq ID No. 4) and NPLFKSATTTVMNPKFAES (Seq ID No. 2) also form part of the present invention. A peptide of the invention may also be extended by, or fused to, heterologous amino acid motifs, sequences or proteins where desired. In this regard, a peptide of the invention should be taken to include fusion peptides or proteins.

A peptide of the invention may be composed of L-amino acids, D-amino acids or a mixture thereof.

It should be appreciated that “Peptide” according to the invention extends to any peptide which is fused with, conjugated to, or otherwise incorporates, a motif which renders it cell-permeable. The motif may allow for active or passive movement of the peptide across or through the cell membrane. The motif may be referred to herein as a cell membrane translocating motif. Such a motif is preferably a peptide-based membrane translocating motif. However, those of skill in the art to which the invention relates will readily recognise motifs of an alternative nature which may effectively provide cell-permeability; for example, motifs that are bound by and internalized by cell-surface receptors, or lipid moieties. The Chariot transfection reagent is designed to transmit biologically active proteins and peptides into living cells, for example.

A peptide-based membrane translocating motif in accordance with the invention will effectively render a peptide cell-permeable, whilst retaining at least a degree of the desired function of said peptide. Those of skill in the art to which the present invention relates will readily appreciate appropriate peptide-based membrane translocating motifs of use in the invention. However, the inventors have found penetratin and a polymer of arginine (as detailed herein after under the heading “Examples”) to be of particular use. Further suitable peptide-based membrane translocating motifs are described in the review by Joliot and Prochiantz-Transduction peptides: from technology to physiology. Nat Cell Biol. 2004; 6(3):189-96 (eg Tat RKKRRQRRR (Seq ID No. 10), Buforin II TRSSRAGLQFPVGRVHRLLRK (Seq ID No. 11), Transportan GWTLNSAGYLLGKINKALAALAKKIL (Seq ID No. 12), MAP (model amphipathic peptide) KLALKLALKALKAALKLA (Seq ID No. 13), K-FGF AAVALLPAVLLALLAP (Seq ID No. 14), Ku70 VPMLK—PMLKE (Seq ID No. 15), Prion MANLGYWLLALFVTMWTDVGLCKKRPKP (Seq ID No. 16), pVEC LLIILRRRIRKQAHAHSK (Seq ID No. 17), Pep-1 KBTWWETWWTEWSQPKKKRKV (Seq ID No. 18), SynB1 RGGRLSYSRRRFSTSTGR (Seq ID No. 19), Pep-7 SDLWEMMMVSLACQY (Seq ID No. 20), HN-1 TSPLNIHNGQKL (Seq ID No. 21).

The amino acid sequences of the peptides of the invention may be modified by substitution of one or more of the amino acids with alternative amino acids, provided the modified peptide retains at least a degree of the desired function of the original peptide. In one preferred embodiment of the invention the amino acid substitution is conservative. Persons skilled in the art will appreciate appropriate conservative amino acid substitutions based on the relative similarity between different amino acids, including the similarity of the amino-acid side chain substituents (for example, their size, charge, hydrophilicity, hydrophobicity and the like). However by way of example, D may be replaced with E, R may be replaced with K, and E may be replaced with D. In another embodiment the amino acid substitution is non-conservative. Persons of skill in the art will appreciate such non-conservative substitutions. However, by way of example, R could be replaced with L. Peptides including amino acid substitutions in accordance with this aspect of the invention will preferably retain at least 50% amino acid sequence similarity, more preferably at least 70%, 80%, 90%, 95% or 99% amino acid sequence similarity to the original peptide.

“Peptides” of the invention may be chemically modified where desirable. For example peptides may be modified by acetylation, glycosylation, cross-linking, disulfide bond formation, cyclization, branching, phosphorylation, conjugation or attachment to a desirable molecule (for example conjugation to bispecific antibodies), acylation, ADP-ribosylation, amidation, covalent attachment of a lipid or lipid derivative, covalent attachment of phosphotidylinositol, demethylation, formation of covalent cross-links, formation of cysteine, formation of pyroglutamate, formylation, gamma-carboxylation, GPI anchor formation, hydroxylation, methylation, myristoylation, oxidation, pegylation, proteolytic processing, prenylation, racemization, conversion from L-isomer to D-isomer, sulfation, or otherwise to mimic natural post-translational modifications, for example. The peptides may also be modified to include one or more non-naturally occurring amino acids, as will be known in the art. Amino acids of a peptide may also be modified by substitution of R groups for other chemical groups as may be known in the art. In addition, amino acids may be substituted with chemical groups which mimic them; for example, benzimidazole is a known mimic of R and 1,4-benzodiazepine a mimic of G-D (see Curr Protein Pept Sci 2005 April; 6(2):151-169. Peptides of the invention may also be modified by arrangement of amino acid groupings from the peptide on a non-peptide scaffold. Considerations for designing such modified peptides are discussed in Curr Protein Pept Sci. 2005 April; 6(2):151-169 (Sillerud and Larson). Amino acids may be modified by attachment of a lipid moiety to facilitate membrane translocation.

The invention should be taken to include pharmaceutically acceptable salts of peptides as well as stereoisomers of peptides. Persons of skill in the art will appreciate such salts and stereoisomers.

Peptides of the invention which have been modified as described herein before (for example, by chemical modification, addition of side groups, addition/inclusion of a cell membrane translocating motif, addition of further amino acids (including heterologous amino acids), inclusion of non-naturally occurring amino acids, substitution of amino acids, substitution of amino acid R groups, salts, isomers, reduction to peptidomimetics, and the like), or by other means known in the art, may be referred to herein as “derivatives” of the peptides.

Use herein of the words “peptide” or “peptides” should be taken to include reference to “derivatives” of such peptides, unless the context requires otherwise. In addition, “peptides” and “derivatives” thereof should be taken to include “prodrugs”, that is peptides or derivatives which are in an inactive form and which are converted to an active form by biological conversion following administration to a subject.

“Derivatives” of the peptides of the invention will retain at least a degree of the desired function of said peptides; that is the ability to modulate the function of β2 integrins (as described herein) and preferably down-regulate, lower or inhibit function. Accordingly, an alternative term for “derivatives” may be “functional derivatives”. The function of a derivative can be assessed, for example, using in vitro cell adhesion assays as described in the “Examples” section herein after. Skilled persons may readily appreciate alternative assays, including in vivo assays in animals.

A peptide of the invention may be purified from natural sources, or preferably derived by chemical synthesis (for example, fmoc solid phase peptide synthesis as described in Fields G B, Lauer-Fields J L, Liu R Q and Barany G (2002) Principles and Practice of Solid-Phase peptide Synthesis; Grant G (2002) Evaluation of the Synthetic Product. Synthetic Peptides, A User's Guide, Grant G A, Second Edition, 93-219; 220-291, Oxford University Press, New York) or genetic expression techniques, methods for which are readily known in the art to which the invention relates. The inventor's contemplate production of a peptide of the invention by an appropriate transgenic animal, microbe, or plant.

To the extent that a peptide of the present invention may be produced by recombinant techniques the invention provides nucleic acids encoding peptides of the invention and constructs or vectors which may aid in the cloning and expression of such nucleic acids. Certain such constructs may also be of use to a therapeutic end as herein after detailed.

Those of general skill in the art to which the invention relates will readily be able to identify nucleic acids which encode peptides of the invention, including desired fusion peptides or proteins, on the basis of the amino acid sequences thereof, the genetic code, and the understood degeneracy therein. However, by way of example: AAT CCC CTT TTC (Seq ID No. 23) (for a peptide having the sequence NPLF), AAG AGC GCC ACC ACG ACG (Seq ID No. 24) (for a peptide having the sequence KSATTT), and AAC CCC AAG TTT (Seq ID No. 25) (for a peptide having the sequence NPKF) are appropriate nucleic acids.

Nucleic acid constructs in accordance with this embodiment of the invention will generally contain heterologous nucleic acid sequences; that is nucleic acid sequences that are not naturally found adjacent to the nucleic acid sequences of the invention. The constructs or vectors may be either RNA or DNA, either prokaryotic or eukaryotic, and typically are viruses or a plasmid. Suitable constructs are preferably adapted to deliver a nucleic acid of the invention into a host cell and are either capable or not capable of replicating in such cell. Recombinant constructs comprising nucleic acids of the invention may be used, for example, in the cloning, sequencing, and expression of nucleic acid sequences of the invention. Additionally, as is herein after detailed, recombinant constructs or vectors of the invention may be used to a therapeutic end.

Those of skill in the art to which the invention relates will recognise many constructs suitable for use in the present invention. However, the inventors contemplate the use of cloning vectors such as pUC and pBluescript and expression vectors such as pCDM8, adeno-associated virus (AAV) or lentiviruses to be particularly useful.

The constructs may contain regulatory sequences such as promoters, operators, repressors, enhancers, termination sequences, origins of replication, and other appropriate regulatory sequences as are known in the art. Further, they may contain secretory sequences to enable an expressed protein to be secreted from its host cell. In addition, expression constructs may contain fusion sequences (such as those that encode a heterologous amino acid motif, for example penetratin, mentioned herein before) which lead to the expression of inserted nucleic acid sequences of the invention as fusion proteins or peptides.

In accordance with the invention, transformation of a construct into a host cell can be accomplished by any method by which a nucleic acid sequence can be inserted into a cell. For example, transformation techniques include transfection, electroporation, microinjection, lipofection, adsorption, and biolistic bombardment.

As will be appreciated, transformed nucleic acid sequences of the invention may remain extrachromosomal or can integrate into one or more sites within a chromosome of a host cell in such a manner that their ability to be expressed is retained.

Any number of host cells known in the art may be utilised in cloning and expressing nucleic acid sequences of the invention. For example, these include but are not limited to microorganisms such as bacteria transformed with recombinant bacteriophage DNA, plasmid DNA or cosmid DNA expression vectors; yeast transformed with recombinant yeast expression vectors; insect cell systems infected with recombinant virus expression vectors (e.g., baculovirus); animal cell systems such as CHO (Chinese hamster ovary) cells using the pEE14 plasmid system; plant cell systems infected with recombinant virus expression vectors (e.g., cauliflower mosaic virus, CaMV; tobacco mosaic virus, TMV) or transformed with recombinant plasmid expression vectors (e.g., Ti plasmid). Those host cells detailed herein after under “Examples” are found to be particularly useful.

A recombinant peptide in accordance with the invention may be recovered from a transformed host cell, or culture media, following expression thereof using a variety of techniques standard in the art. For example, detergent extraction, sonication, lysis, osmotic shock treatment and inclusion body purification. The protein may be further purified using techniques such as affinity chromatography, ion exchange chromatography, filtration, electrophoresis, hydrophobic interaction chromatography, gel filtration chromatography, and chromatofocusing.

As mentioned herein before, a peptide of the invention may be in the form of a fusion peptide or protein; for example, a peptide of the invention attached to a peptide-based membrane translocating motif, or alternatively, or in addition, a motif which may aid in subsequent isolation and purification of the peptide (for example, ubiquitin, his-tag, or biotin). Means for generating such fusion peptides are known in the art to which the invention relates, and include chemical synthesis and techniques in which fusion peptides are expressed in recombinant host cells, as mentioned herein. The inventors contemplate Strep-tag (Sigma-Genosys), Impact™ system (New England Biolabs), his-tag, and the eg pMAL™-p2 expression system (New England BioLabs), to be particularly useful in the present instance. In addition, fusion tags of use in recombinant protein expression and purification have been described by R. C. Stevens. “Design of high-throughput methods of protein production for structural biology” Structure, 8, R177-R185 (2000).

Membrane translocating motifs may also be fused, conjugated or otherwise incorporated in or attached to a peptide by alternative means known in the art to which the invention relates. For example, where cell-permeabilising moieties comprise an entire protein, fatty acids and/or bile acids, such molecules may be linked to the active peptide by an amino acid bridge, or by a non-peptidyl linkage.

A peptide of the invention or derivative thereof may be simultaneously joined to two tags, where one tag allows for cell secretion (eg signal peptide), and another tag renders the peptide cell-permeable. In this scenario the peptide or derivative thereof could be produced and secreted by a non-leukocyte to be subsequently taken up by a leukocyte. This could be advantageous for instance where one may wish parenchymal or endothelial cells within an inflamed tissue to secrete the peptide to inhibit the adhesion of infiltrating leukocytes.

### Compositions and Methods of Treatment

Inasmuch as the present invention relates to the modulation of integrin β2 function, including the treatment of inflammatory disorders, it also provides a pharmaceutical composition comprising agents of the invention in association with one or more pharmaceutically acceptable diluents, carriers and/or excipients.

As used herein, the phrase “pharmaceutically acceptable diluents, carriers and/or excipients” is intended to include substances that are useful in preparing a pharmaceutical composition, may be co-administered with an appropriate agent for example a peptide, derivative thereof, nucleic acid encoding said peptide, or construct comprising same, of the invention while allowing the agent to perform its intended function, and are generally safe, non-toxic and neither biologically nor otherwise undesirable. Pharmaceutically acceptable diluents, carriers and/or excipients include those suitable for veterinary use as well as human pharmaceutical use. Examples of pharmaceutically acceptable diluents, carriers and/or excipients include solutions, solvents, dispersion media, delay agents, emulsions and the like.

In addition to standard diluents, carriers and/or excipients, a pharmaceutical composition in accordance with the invention may be formulated with additional constituents, or in such a manner, so as to enhance the activity of an agent of the invention, or help protect the integrity of such agents. For example, the composition may further comprise constituents which provide protection against proteolytic degradation, enhance bioavailability, decrease antigenicity, or enable slow release upon administration to a subject. For example, slow release vehicles include macromers, poly(ethylene glycol), hyaluronic acid, poly(vinylpyrrolidone), or a hydrogel.

Furthermore, cell permeability of an agent of the invention may be achieved, or facilitated, through formulation of the composition.

Additionally, it is contemplated that a pharmaceutical composition in accordance with the invention may be formulated with additional active ingredients which may be of benefit to a subject in particular instances. Persons of ordinary skill in the art to which the invention relates will readily appreciate suitable additional active ingredients having regard to the description of the invention herein and the nature of a particular disorder to be treated, for example. As a general example, antibodies, small molecule inhibitors, immunosuppressors, pharmaceutical drugs (eg steroids), may be used.

In one embodiment, the present invention also pertains to methods for the treatment of inflammatory disorders comprising at least the step of administering to a subject in need thereof a therapeutically effective amount of an agent of the invention or a pharmaceutical composition comprising same.

It should be appreciated that peptides (and derivatives thereof) of the invention may be administered and formulated as pro-drugs, which are converted to active agents following administration.

As used herein, a “therapeutically effective amount”, or an “effective amount” is an amount necessary to at least partly attain a desired response.

The inventors contemplate administration of an agent of the invention, or pharmaceutical compositions comprising one or more agents of the invention, by any means capable of delivering such agents to leukocytes at a target site within the body of a subject; a “target site” is a site at which an inflammatory event has, or is predicted to, occur, or a site which may otherwise benefit from the delivery of said agent(s). By way of example, agents of the invention may be administered as pharmaceutical compositions by one of the following routes: oral, topical, systemic (eg. transdermal, intranasal, or by suppository), parenteral (eg. intramuscular, subcutaneous, or intravenous injection), by administration to the CNS (eg. by intraspinal or intracisternal injection); by implantation, and by infusion through such devices as osmotic pumps, transdermal patches, and the like. Further examples may be provided herein after. Skilled persons may identify other appropriate administration routes.

In accordance with such modes of administration, and the suitable pharmaceutical excipients, diluents and/or carriers mentioned herein before, compositions of the invention may be converted to customary dosage forms such as solutions, orally administrable liquids, injectable liquids, tablets, coated tablets, capsules, pills, granules, suppositories, trans-dermal patches, suspensions, emulsions, sustained release formulations, gels, aerosols, powders and immunoliposomes. Additionally, sustained release formulations may be utilised. The dosage form chosen will reflect the mode of administration desired to be used. Particularly preferred dosage forms include orally administrable tablets, gels, pills, capsules, semisolids, powders, sustained release formulation, suspensions, elixirs, aerosols, ointments or solutions for topical administration, and injectable liquids. Further specific examples will be provided herein after.

As will be appreciated, the dose of an agent or composition administered, the period of administration, and the general administration regime may differ between subjects depending on such variables as the severity of symptoms of a subject, the type of disorder to be treated, the mode of administration chosen, and the age, sex and/or general health of a subject.

Data obtained from cell culture assays and animal studies can be used in formulating a range of dosages for use in humans. The dosage may vary within this range depending upon the dosage form employed and the route of administration utilized. For any compound used in the method of the invention, the therapeutically effective dose can be estimated initially from cell culture assays. A dose may be formulated in cell cultures or animal models to achieve a cellular concentration range that includes the IC50 (i.e., the concentration of the test compound that achieves a half-maximal inhibition of symptoms) as determined in cell culture. Such information can be used to more accurately determine useful doses in humans. The exact formulation, route of administration and dosage can be chosen by the individual physician in view of the patient's condition. (See, e.g., Fingl et al., 1975, In: The Pharmacological Basis of Therapeutics, Ch. 1, p. 1).

Specific examples of compositions and modes of administration relevant to 1) peptides, and 2) nucleic acids are now provided. These are given by way of example only.

### Peptide Compositions and Modes of Administration

Those skilled in the art of peptide-based treatments will readily appreciate a variety of pharmaceutically acceptable diluents, carriers and/or excipients which may be employed in compositions of the invention comprising one or more peptides. By way of example, suitable liquid carriers, especially for injectable solutions, include water, aqueous saline solution, aqueous dextrose solution, and the like, with isotonic solutions being preferred for intravenous, intraspinal, and intracisternal administration. Diluents, carriers and/or excipients may be chosen to enhance peptide stability. For example, one or more of the following may be used: buffer(s), blocking agent(s), solvent(s), salt(s), chelator(s), detergent(s), and preservative(s). Stabilizing diluents for polypeptides and antigens are described for example in U.S. Pat. No. 6,579,688.

As mentioned herein before, peptides of the invention may be formulated to allow for slow release. Pharmaceutical compositions for prolonged peptide release and preparation method are described for example in U.S. Pat. Nos. 6,503,534 and 6,482,435, and 6,187,330, and 6,011,011. In addition, to prolong the in vivo half-life of proteins and to reduce their antigenicity proteins may be conjugated to soluble synthetic polymers, in particular poly(ethylene glycol), poly(vinyl pyrrolidone), poly(vinyl alcohol), poly(amino acids), divinylether maleic anhydride, ethylene-maleic anhydride, N-(2-hydroxypropyl)methacrylamide and dextran. Methods for synthesis of polymer bio-active conjugates are described for example in U.S. Pat. No. 6,172,202. Peptides may also be delivered via implants as described in U.S. Pat. No. 6,077,523.

Furthermore, while a peptide of the invention may be rendered cell-permeable by fusion or conjugation to an appropriate membrane translocating motif, cell permeability may alternatively be achieved, or further be facilitated, through formulation of the composition. Pharmaceutical formulation of a therapeutic polypeptide together with a permeation-enhancing mixture to enhance bioavailability is described for example in U.S. Pat. No. 6,008,187.

Methods of formulating a peptide composition of the invention will be readily appreciated by persons of ordinary skill in the art to which the invention relates. Nonetheless, guidance may be found in Gennaro A R: Remington: The Science and Practice of Pharmacy, 20th ed., Lippincott, Williams & Wilkins, 2.000.

As will be appreciated, the dose of a peptide (or composition comprising same) administered, the period of administration, and the general administration regime may differ between subjects depending on such variables as mentioned herein before. However, by way of general example, the inventors contemplate administration of from approximately 30 μg to 300 mg per kilogram (mg/Kg) mass of the animal, for example, 0.3 to 30 mg/Kg, with lower doses such as 0.003 to 0.3 mg/Kg, e.g. about 0.03 mg/Kg, being appropriate for administration through the cerebrospinal fluid (for example, which may be appropriate in treatment of encephalitis including multiple sclerosis) such as by intracerebroventricular administration, and higher doses such as 3 to 300 mg/Kg, e.g. about 30 mg/Kg, being appropriate for administration by methods such as oral, systemic (eg. transdermal), or parenteral (e.g. intravenous) administration.

### Gene Therapy—Compositions and Modes of Administration

As mentioned herein before, methods of the invention may involve the administration of nucleic acids encoding peptides of the invention and/or constructs comprising same. The use of such nucleic acid techniques may be referred to herein as “gene therapy”.

Any of the methods for gene therapy available in the art can be used according to the present invention. Exemplary methods are described below. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215).

Methods commonly known in the art of recombinant DNA technology which can be used in generating appropriate constructs or vectors are described generally herein before and more specifically for example in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In one aspect, a composition comprising at least nucleic acid sequences encoding a peptide of the invention in expression vectors are administered to suitable hosts. The expression of nucleic acid sequences encoding a peptide of the invention may be optimized by enlarging the sequence either by including repeats of the peptide sequence or including flanking heterologous sequences to enable the sequence to be expressed, and processed by the translational machinery. The sequence may be fused with a signal peptide and cell-permeable peptide to allow for secretion, and cell uptake. The expression of nucleic acid sequences encoding a peptide of the invention may be regulated by any inducible, constitutive, or tissue-specific promoter known to those of skill in the art. In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription. In a particular embodiment, nucleic acid molecules encoding a peptide of the invention are flanked by regions that promote homologous recombination at a desired site in the genome, thus providing for intrachromosomal expression of said coding regions (Koller and Smithies, 1989, Proc. Natl. Acad. Sci. USA 86:8932-8935; Zijlstra et al., 1989, Nature 342:435-438).

Delivery of the nucleic acids into a patient may be either direct, in which case the patient is directly exposed to the nucleic acid molecules or constructs containing them, or indirect, in which case, cells are first transformed with the nucleic acid molecules in vitro to express secretable cell-permeable forms of the peptide, and then transplanted into the patient. These two approaches are known, respectively, as in vivo or ex vivo gene therapy.

In a specific embodiment, the nucleic acid molecules are directly administered in vivo, where they are expressed to produce the encoded product. This can be accomplished by any of numerous methods known in the art; for example, they may be constructed as part of an appropriate nucleic acid expression vector and administered so that they become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), and the like.

In another embodiment, nucleic acid-ligand complexes can be formed in which the ligand comprises a fusogenic viral peptide to disrupt endosomes, allowing the nucleic acid molecules to avoid lysosomal degradation.

In yet another embodiment, the nucleic acid molecules can be targeted in vivo for cell specific uptake and expression, by targeting a specific receptor (as described for example in WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.); and, WO 93/20221 dated Oct. 14, 1993 (Young)). Alternatively, the nucleic acid molecules can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, viral vectors are used to express nucleic acid sequences. Persons of skill in the art to which the invention relates may appreciate a variety of suitable viral vectors having regard to the nature of the invention described herein. However, by way of example, a retroviral vector can be used (see Miller et al., 1993, Meth. Enzymol. 217:581-599). Such retroviral vectors have deleted retroviral sequences that are not necessary for packaging of the viral genome and integration into host cell DNA. More detail about retroviral vectors can be found in Boesen et al., 1994, Biotherapy 6:291-302, for example. Other references illustrating the use of retroviral vectors in gene therapy include, for example: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. In Genetics and Devel. 3:110-114.

Another example of a suitable viral vector of use in gene therapy techniques applicable to the invention includes adenoviruses. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146). AAV present the most preferable viral vectors for use in the present invention. AAV vectors have been reported to lead to persistent (>6 months) expression of a transgene in both gut epithelial cells and hepatocytes, resulting in long-term phenotypic recovery in a diabetic animal model (Xu, R A et al., 2001, Peroral transduction of diffuse cells and hepatocyte insulin leading to euglycemia in diabetic rats, Mol Ther 3:S180; During, M J et al., 1998, Peroral gene therapy of lactose intolerance using an adeno-associated virus vector, Nature Med. 4:1131-1135; During M J et al., 2000, An oral vaccine against NMDAR1 with efficacy in experimental stroke and epilepsy, Science 287:1453-1460). AAV is a nonpathogenic, helper-dependent member of the parvovirus family with several major advantages, such as stable integration, low immunogenicity, long-term expression, and the ability to infect both dividing and non-dividing cells. It is capable of directing long-term transgene expression in largely terminally differentiated tissues in vivo without causing toxicity to the host and without eliciting a cellular immune response to the transduced cells (Ponnazhagan S et al., 2001, Adeno-associated Virus for Cancer Gene Therapy, Cancer Res 61:6313-6321; Lai C C et al., 2001, Suppression of choroidal neovascularization by adeno-associated virus vector expressing angiostatin, Invest Opthalmol V is Sci 42(10):2401-7; Nguyen J T et al., 1998, Adeno-associated virus-mediated delivery of antiangiogenic factors as an antitumor strategy, Cancer Research 58:5673-7).

In a preferred embodiment of the invention, the cells into which a nucleic acid can be introduced for purposes of gene therapy are leukocytes. However, any desired, available cell type, could be used, especially where the nucleic acid is adapted to express a peptide to be secreted from the cell and subsequently taken up by a leukocyte. For example, the nucleic acid may be introduced into epithelial cells, endothelial cells, keratinocytes, fibroblasts, muscle cells, hepatocytes; leukocytes such as T lymphocytes, B lymphocytes, monocytes, and macrophages.

As mentioned herein before, nucleic acids and nucleic acid constructs of use in this aspect of the invention may be formulated into appropriate compositions in association with one or more pharmaceutically acceptable diluents, carriers and/or excipients. Skilled persons will readily appreciate such suitable diluents, carriers and/or excipients. However, by way of specific example, suitable liquid carriers, especially for injectable solutions, include water, aqueous saline solution, aqueous dextrose solution, and the like, with isotonic solutions being preferred.

The nucleic acids, constructs and viruses may be formulated to help assist in delivery, or protect the integrity of the nucleic acid in vivo. For example, they may be formulated into liposomes, microparticles, microcapsules, or recombinant cells, or as a part of appropriate viral vectors. They may also be formulated-to-make-use-of-delivery by receptor-mediated endocytosis (see, e.g., Wu and Wu, J. Biol. Chem. 262:4429-4432 (1987)). Lipid Polycation DNA (LPD) may be employed in which DNA is condensed prior to encapsulation in the lipid (as used by Targeted Genetics Corporation, Seattle, Wash., USA).

Specific examples of methods of administering a gene-therapy-based composition of the invention include, but are not limited to, parenteral administration (e.g., intradermal, intramuscular, intraperitoneal, intravenous and subcutaneous), epidural, and mucosal (e.g., intranasal and oral routes). In a specific embodiment, prophylactic or therapeutic compositions of the invention are administered intramuscularly, intravenously, or subcutaneously. The composition may be administered by any convenient route, for example by infusion or injection, by absorption through epithelial or mucocutaneous linings (e.g., oral mucosa, rectal and intestinal mucosa, etc.) and may be administered together with other biologically active agents. Administration can be systemic or local. Systemic gene-therapy via intravenous administration provides a preferable mode of administration.

### Methods of Identification of Functional Interactor Molecules

Methods of identifying β2 integrin functional interactor molecules, including interference molecules (such as aptamers), of the peptides of the invention, will generally comprise at least the step of bringing a potential functional interactor in contact with a peptide or derivative thereof of the invention and observing whether or not binding occurs. For example, such molecules can be identified by “pull-down” assays whereby a peptide of the invention is immobilised on a matrix eg Sepharose beads and used to affinity isolate interactors from a cell lysate. The interactors can be electrophoresed on an SDS-gel and identified by Western blotting with mAbs against candidate interactors, or the interactors are identified directly by mass spectroscopy. The peptides of the invention may be immobilised on a column, and used to affinity purify interactors. BiaCORE technology based on surface plasmon resonance can be used to establish or characterise molecular interactions.

It should be appreciated that the peptides and derivatives thereof may also be used to screen libraries of molecules for potential interactors; for example aptamer libraries (such as those of Archemix, Cambridge, Mass.) and libraries of synthetic antibodies (for example, HuCAL®b antibody libraries (Morphosys AG, Martinsried/Planegg, Germany)).

Once binding to a peptide (or derivative thereof) of the invention has been established, the function of a candidate molecule, can be assessed, for example, using in vitro cell adhesion assays as described in the “Examples” section herein after. Interactors can also be over-expressed or inhibited (eg with antisense, RNAi etc) to determine whether they regulate the function of β2 integrins. Skilled persons may readily appreciate alternative assays, including in vivo assays in animals.

“Interference molecules” will exhibit at least some ability to disrupt or inhibit the activity and function of a β2 integrin. Preferably they will disrupt or prevent the interaction of β2 integrins with their ligands.

### Interference Molecules

Nucleic acid aptamers directed to the peptides of the invention may be developed using the following approaches. The SELEX technique (systematic evolution of ligands by exponential enrichment) is an anti-protein approach in which nuclease-resistant DNA or RNA aptamers are selected by their ability to bind their protein targets with high affinity and specificity of the same range as antibodies (for example, J. Hesselberth, M. P. Robertson, S. Jhaveri and A. D. Ellington Mol. Biotech. 74 (2000), pp. 15-25; A. D. Ellington and J. W. Szostak Nature 346 (1990), pp. 818-822; C. Tuerk and L. Gold Science 249 (1990), pp. 505-510). Further, a vaccinia virus-based RNA expression system has enabled high-level cytoplasmic expression of RNA aptamers directed against the intracellular domain of the beta2 integrin LFA-1. Aptamers can be prepared and screened, for example, in accordance with the methodology described in Blind M, Kolanus W, Famulok M. Cytoplasmic RNA modulators of an inside-out signal-transduction cascade. Proc Natl Acad Sci U S A. 1999; 96(7): 3606-3610.

Peptides or derivatives thereof in accordance with the invention may be used as antigens for the production of antibodies. Such antibodies may have specific application in experimental studies of the functions of β2 integrins, or as prophylactic or therapeutic reagents when rendered cell-permeable. Anti-idiotypic antibodies raised against antibodies that recognise peptides of the invention may be used to identify potential interactors, or for therapy (McCarthy H, Ottensmeier C H, Hamblin T J, Stevenson F K. Anti-idiotype vaccines. Br J Haematol. 2003; 123(5):770-81).

The term “antibody” should be understood in the broadest possible sense and is intended to encompass, for example, intact monoclonal antibodies, polyclonal antibodies, and derivatives of such antibodies; for example, hybrid and recombinant antibodies (for example, humanised antibodies, diabodies, triabodies, tetrabodies and single chain antibodies) (Le Gall F, Kipriyanov S M, Moldenhauer G, Little M. Di-, tri-, and tetrameric single chain Fv antibody fragments against human CD19; effect of valency on cell binding. FEBS Lett. 1999; 453(1-2):154-168) and antibody fragments so long as they exhibit the desired biological activity. An antibody may also be modified so as to render it cell-permeable (a “Transbody”). This may be achieved using the membrane translocation motif technology described herein before. In addition, the methodology described by Heng and Cao (Med. Hypotheses. 2005; 64(6):1105-8) may be used.

Antibody “fragments” is intended to encompass a portion of an intact antibody, generally the antigen binding or variable region of the antibody. Examples of antibody fragments include Fab, Fab′ F(ab′)₂, and Fv fragments. Those of ordinary skill in the art to which the invention relates will recognise methods to generate such antibody fragments. However, by way of general example proteolytic digestions of intact antibodies may be used, or the fragments may be directly produced via recombinant nucleic acid technology.

“Humanised” antibodies are essentially hybrid or chimeric antibodies containing domains derived from human sources and domains derived from the animal in which an antibody may have been generated. In the present case, they are either fully-human or mouse/human-hybrid antibodies. Humanised antibodies in accordance with the invention will generally comprise the mouse CDR (complementarity determining region or antigen binding site) of an antibody against of peptide of the invention fused to appropriate human antibody domains or regions necessary to form a functional antibody, for example. Humanization of murine antibodies can be achieved using techniques known in the art, for example by epitope-guided selection (Wang et al, 2000). The methods of Jones et al (1986), or Maynard and Georgiou (2000) provide further examples.

Humanisation of antibodies may help reduce the immunogenicity of the antibodies of the invention in humans for example. Reduced immunogenicity can be obtained by transplanting murine CDR regions to a homologous human P sheet framework (termed CDR grafting; refer to Riechmann et al 1988 and Jones et al 1986).

Those of skill in the art to which the invention relates will appreciate the terms “diabodies” and “triabodies”. These are molecules which comprise a heavy chain variable domain (VH) connected to a light chain variable domain (VL) by a short peptide linker that is too short to allow pairing between the two domains on the same chain. This promotes pairing with the complementary domains of one or more other chain encouraging the formation of dimeric or trimeric molecules with two or more functional antigen binding sites. The resulting antibody molecules may be monospecific or multispecific (eg bispecific in the case of diabodies). Such antibody molecules may be created from two or more of the antibodies of the present invention using methodology standard in the art to which the invention relates; for example, as described by Holliger et al (1993), and Tomlinson and Holliger (2000).

The production of antibodies in accordance with the invention may be carried out according to standard methodology in the art. For example, in the case of the production of polyclonal antibodies the method of Diamond et al (1981) may be used. Monoclonal antibodies may be prepared, for example, as described in Current Protocols in Immunology (1994, published by John Wiley & Sons and edited by: John E. Coligan, Ada M. Kruisbeek, David H. Margulies, Ethan M. Shevach, Warren Strober), by Winter and Milstein (1991), or in “Monoclonal Antibody Production Techniques and Applications”, Marcel Dekker Inc.

Production of an antibody or derivative thereof may also be achieved using standard recombinant techniques known in the art, and discussed previously herein. It will be appreciated that nucleic acids encoding an antibody, and thus suitable for recombinant production of the antibody, may be identified by isolating and sequencing nucleic acids from an appropriate hybridoma, or by having regard to the amino acid sequence of the antibody and knowledge of the genetic code and degeneracy therein. The amino acid sequence of an antibody of the invention may be determined using standard methodology; for example, the technique of Edman degradation and HPLC or mass spectroscopy analysis (Hunkapiller et al, 1983), may be used.

The inventors consider recombinant techniques to be a preferable means of producing antibodies on a commercial scale for therapeutic applications.

Antibodies or derivatives thereof may be formulated into pharmaceutical compositions in a similar manner as described herein before, particularly in relation to formulation of the peptides of the invention (see in particular the sections entitled “compositions and methods of treatment” and “peptide compositions and modes of administration”). Antibodies may also be administered in accordance with the principles described in those sections. Improved delivery methods for antibodies include controlled-release and local delivery strategies as described, for example, by Grainger (in “Controlled-release and local delivery of therapeutic antibodies”, Expert Opin Biol Ther. 2004 July; 4(7): 1029-44).

Antibodies may also be delivered to a subject in the form of “intrabodies”, or nucleic acid constructs which are adapted to express the antibodies in desired cells following plasmid or viral delivery, for example. Inasmuch as this is the case, appropriate nucleic acids can be formulated into acceptable pharmaceutical compositions and administered as herein before described in the sections entitled “compositions and methods of treatment” and “gene-therapy—compositions and modes of administration. Stocks (in Intrabodies: production and promise. Drug Discov Today. 2004 Nov. 15; 9(22):960-6.) provides further guidance on the production of “intrabodies”.

### Kits

The agents of the invention may be used in kits suitable for modulating the function of integrin β2 or for the treatment of integrin β2-mediated inflammatory disorders. Such kits will comprise at least an agent of the invention in a suitable container. The agent may be formulated suitable for direct administration to a subject (for example, as an agent or pharmaceutical composition). Alternatively, the kit may comprise the agent in one container and a pharmaceutical carrier composition in another; the contents of each container being mixed together prior to administration. The kit may also comprise additional agents and compositions in further separate containers as may be necessary for a particular application. Further, kits of the invention can also comprise instructions for the use and administration of the components of the kit.

Any container suitable for storing and/or administering a pharmaceutical composition may be used in a kit of the invention. Suitable containers will be appreciated by persons skilled in the art. By way of example, such containers include vials and syringes. The containers may be suitably sterilised and hermetically sealed.

### EXAMPLES

### Materials and Methods

### Cell Lines and Synthetic Peptides

The human T lymphoma cell line H9, was purchased from the American Type Culture Collection, Rockville, Md. It was cultured at 37° C. in 1640 medium supplemented with 50 U/ml penicillin, 50 μg/ml streptomycin, 200 μg/ml L-glutamine, 10% (v/v) FCS and 0.05 mM β-mercaptoethanol. All synthetic peptides were custom made by Mimotopes Pty Ltd., Victoria, Australia. The β2cyt peptides were N-terminally fused during synthesis to biotinylated penetratin (RQIKIWFQNRRMKWKK—Seq ID No. 22) or to a biotinylated D-isomeric form of an R9 polymer (Seq ID No. 32) to render them cell-permeable.

### Recombinant ICAM-1-Fc Chimeras

The soluble ICAM-1-Fc chimera was produced using the glutamine synthetase gene amplification system. The extracellular portions of human ICAM-1 fused to the Fc domain of human IgG1 were expressed from the pEE14 vector (kindly provided by Dr Chris Bebbington, Celltech Ltd, UK) in CHO K1 cells as described previously (18).

### Peptide Internalization and Visualization

Biotinylated peptides were added to the cells in serum-free RPMI 1640 medium for 30 min to 2 h at 37° C. or room temperature. The cells were washed twice with PBS, resuspended into 1% FCS in PBS, and cytocentrifuged onto glass slides. Cytospin smears were fixed with 4% paraformaldehyde in PBS for 15 min at room temperature (RT), washed twice with PBS and permeabilized in PBS containing 0.2% Triton X-100. Biotinylated peptides were detected by incubating the cytospins with streptavidin-FITC (Sigma, Mo.) for 45 min at RT, and visualized using either a Leica TCS 4D confocal laser microscope, or Nikon E600 fluorescence microscope. Images were processed using Leica Scanware™ 4.2 A software and Adobe Photoshop 5.0.

### Cell Adhesion Assay

Lab-Tek 16-well glass slides (Nunc) or flat-bottom 96-well plates (Nunc Maxisorp) were coated overnight at 4° C. with purified ICAM-1-Fc at 10 μg/well in 100 μl of 0.1 M carbonate buffer (pH 9.5). After washing plates were blocked with 100% heat-inactivated FCS, and washed with Hanks balanced salt solution (HBSS) containing 10 mM Hepes, 2 mM Ca²⁺ and 2 mM Mn²⁺. H9 cells were either left unlabeled or labeled with the fluorescent dye chloromethyl fluorescein diacetate (CMFDA; Molecular Probes, Oregon). For peptide inhibition studies, H9 cells were preincubated with peptide in serum-free medium for 30 min at 37° C. and activated by suspension in an Mn²⁺-containing buffer (HBSS: 10 mM Hepes containing 2 mM Ca²⁺, 2 mM Mn²⁺, and 2% FCS). Human IgG1 Ab (10 μg/ml; Sigma) was added to prevent nonspecific capture of cells to the Fc portion of VCAM-1. Cells were checked for viability by trypan blue exclusion, added to wells (10⁶cells/well), and incubated for 30 min at 37° C. in a humidified atmosphere of 5% CO₂. Non-adherent cells were removed by inverse centrifugation of the plates at 70×g for 5 min followed by gentle pipette washing. The number of unlabeled adherent cells was determined by counting the number of adherent cells in four independent fields at 100× magnification under an inverted microscope. The fluorescence of CMFDA-labeled adherent cells was measured using a VICTOR1420 multilabel counter (Wallac). Representative data are reported as mean±SD of two independent experiments performed in duplicate.

### Results

### Three Short Peptides Near the C-Terminus of the β2 Subunit Cytoplasmic Tail Inhibit αLβ2-Mediated T Cell Adhesion

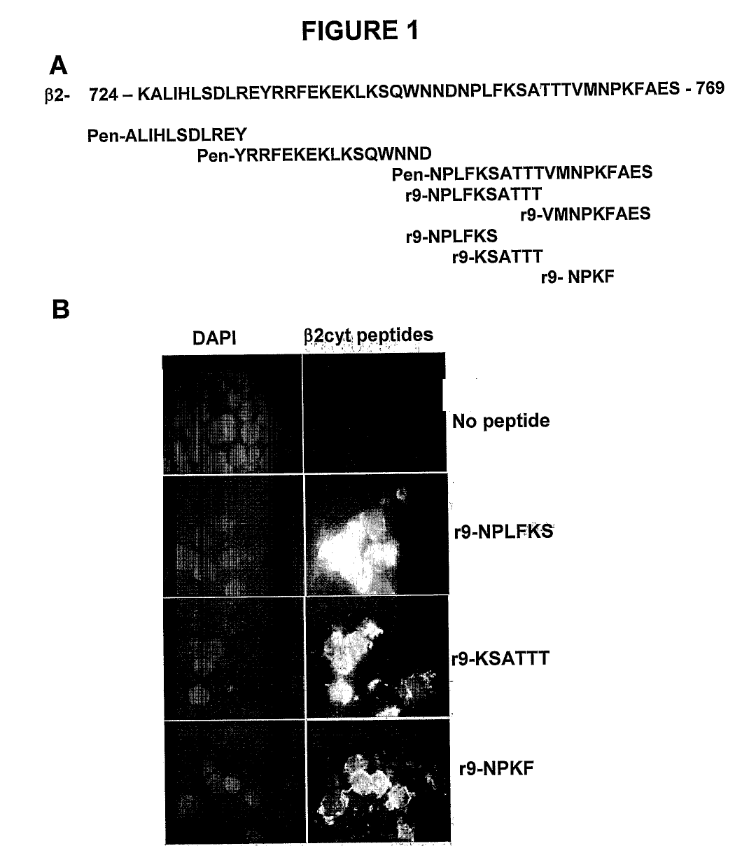

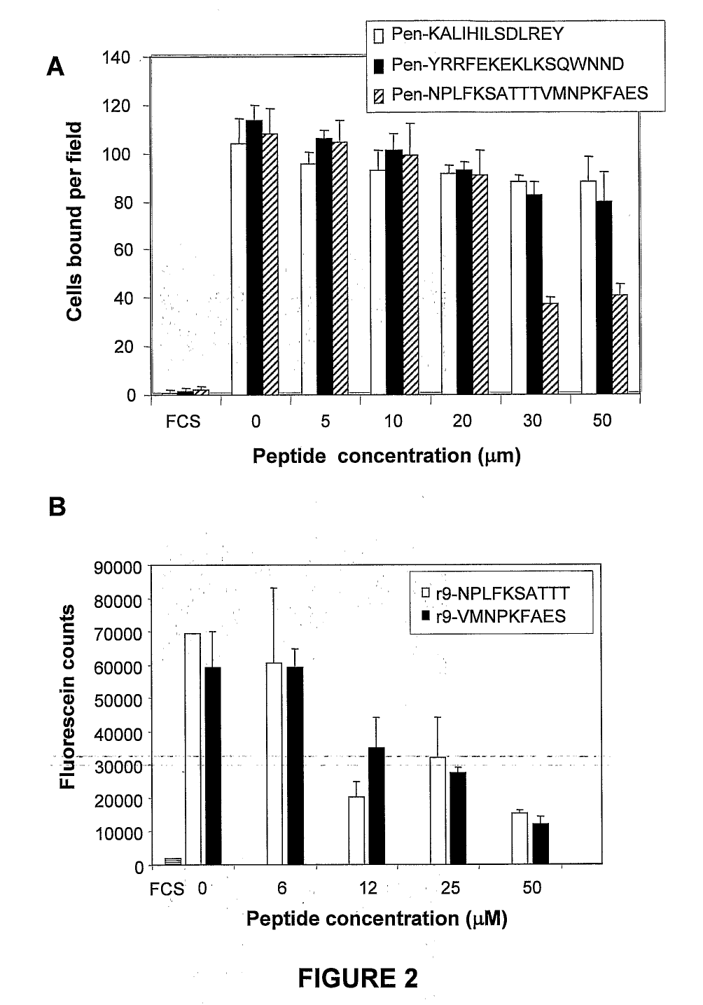

Three peptides 724-KALIHLSDLREY-735 (Seq ID No. 1), 735-YRRFEKEKLKSQWNND-750 (Seq ID No. 30), and 751-NPLFKSATTTVMNPKFAES-769 (Seq ID No. 2) encompassing the entire cytoplasmic domain of the β2 subunit (β2cyt) were fused at their N-termini to penetratin. All three fusion peptides were readily taken up by the human cell line H9 (αLβ2⁺) derived from a cutaneous T cell lymphoma (derivative of HUT78) (FIG. 1). When tested for their abilities to block αLβ2-mediated adhesion of Mn⁺⁺-activated H9 cells to ICAM-1, only peptide Pen-NPLFKSATTTVMNPKFAES (Seq ID No. 2) from the C-terminal end of the β2 subunit tail significantly (p<0.001) inhibited cell adhesion (FIG. 2A). Maximal inhibition of adhesion (64%) was achieved at a concentration of 30 μM. The NPLFKSATTTVMNPKFAES (Seq ID No. 2) peptide was divided into two in order to sublocalize the bioactive sequence, giving the peptides NPLFKSATTT (Seq ID No. 3) and VMNPKFAES (Seq ID No. 4) which were fused to r9 to render them cell-permeable. The two peptides displayed similar abilities at inhibiting the adhesion of Mn⁺⁺-activated H9 cells to ICAM-1, causing ˜50% inhibition at 25 μM, and ˜80% inhibition at 50 μM (FIG. 2B).

To further sublocalize the bioactive regions of the NPLFKSATTT (Seq ID No. 3) and VMNPKFAES (Seq ID No. 4) peptides, a further three peptides (r9-NPLFKS (Seq ID No. 5), r9-KSATTT (Seq ID No. 6), and r9-NPKF (Seq ID No. 7)) were synthesized as fusions with the r9 carrier peptide. The NPKF (Seq ID No. 7) motif in the VMNPKFAES (Seq ID No. 4) peptide was chosen since it resembled the NPLF (Seq ID No. 8) motif in the NPLFKSATTT (Seq ID No. 3) peptide. Each of the latter peptides inhibited the adhesion of H9 cells to ICAM-1 by 24%, 80%, and 82%, respectively at 6 μM (FIG. 2C), and almost completely blocked adhesion at 100 μM. Thus, the hexamer NPLFKS (Seq ID No. 5) and its core motif NP(L/K)F and the hexamer KSATTT (Seq ID No. 6) representing adjacent bioactive sites in the C-terminal region of the tail of the β2 subunit participate in signaling events required for activation of the adhesiveness of β2-integrin.

### Discussion

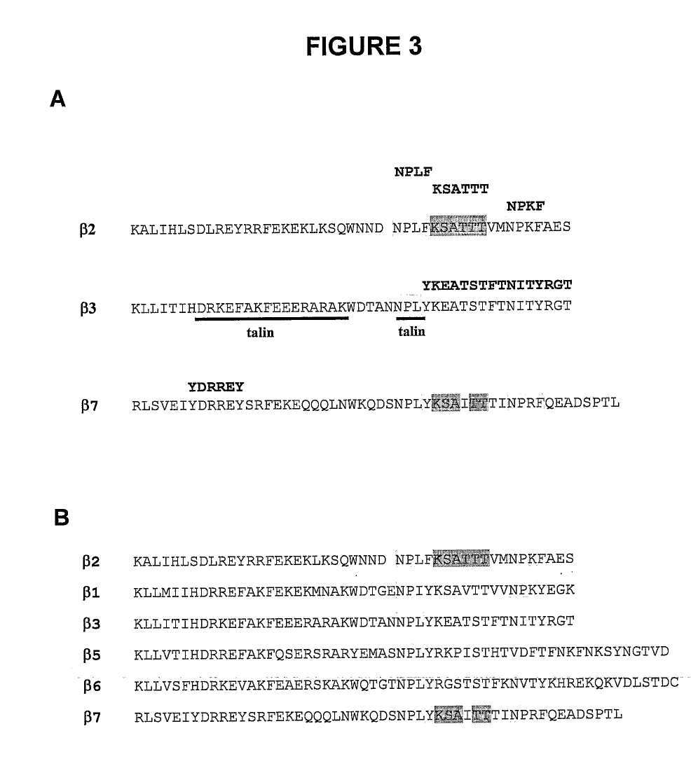

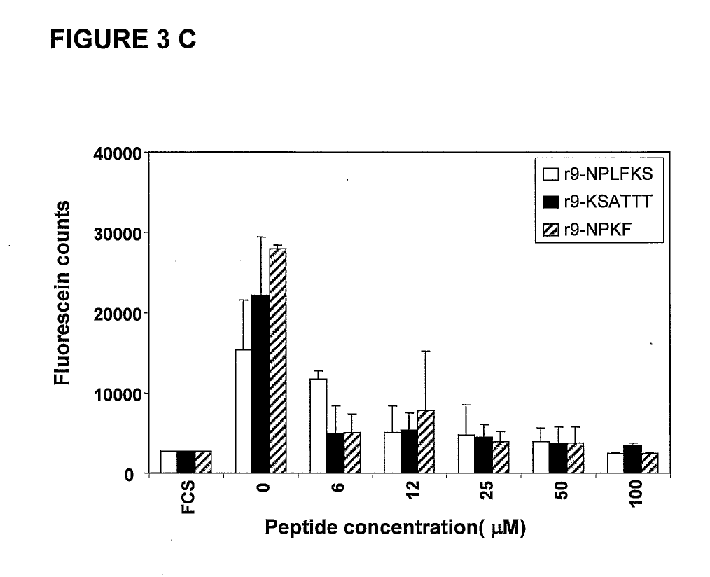

The results reveal that for the β2 subunit, two small adjacent peptide motifs in the C-terminal region of the cytoplasmic tail play key roles in mediating the adhesive function of the β2 integrins. The core sequence for one of these motifs (NPxF—Seq ID No. 9) in β2 is repeated near the C-terminus of the β2 subunit tail (FIG. 3).

The cytoplasmic tails of integrin β3 subunits are generally reasonably well conserved (FIG. 3). Sequences closely related to the three 751-NPLF-754 (Seq ID No. 8), 755-KSATTT-760 (Seq ID No. 6), and 763-NPKF-766 (Seq ID No. 7) CARDs identified by the inventors in β2cyt can be found in a few other integrin β subunits. Whereas the NPxY (Seq ID No. 31) motif is found in six other β subunits, the NPxF (Seq ID No. 9) motif is unique to the β2 subunit. Motifs related to the KSATTT (Seq ID No. 6) CARD can also be found in the P1 (KSAVTT—Seq ID No. 26) and β7 (KSAITTT—Seq ID No. 27) subunits (FIG. 3).

Studies have identified CARDs in the β1, β3 and β7 subunits that are completely unrelated to those reported here for the β2 subunit, suggesting that different integrins display specificity in regards to which cytoplasmic tail motifs and signalling molecules are obligatory for activation of adhesiveness (FIG. 3). Peptides containing NPxY/F motifs from the β3 (NPLY—Seq ID No. 28) and β7 (NPLY (Seq ID No. 28) and NPRF (Seq ID No. 29)) tails fail to block α4β7-mediated T cell adhesion, suggesting the β2 subunit is unique in its dependence on this motif for activation. Similarly, a peptide from the P7 subunit containing the KSATTT-related motif KSAITTT fails to inhibit α4β7-mediated T cell adhesion indicating the KSATTT CARD is functionally unique to the β2 subunit.

The invention has been described herein, with reference to certain preferred embodiments, in order to enable the reader to practice the invention without undue experimentation. However, a person having ordinary skill in the art to which the invention relates will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in the field of endeavour to which the invention relates.

Throughout this specification, and any claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### REFERENCES

1. Krissansen, G. W. Integrin superfamily. The Encyclopedia of Life Sciences. MacMillan Press, 2000.
2. Yauch, R. L., Felsenfeld, D. P., Kraeft, S-K., Chen, L. B., Sheetz, M. P., and Hemler, M. E. Mutational evidence for control of cell adhesion through integrin diffusion/clustering, independent of ligand binding. J. Exp. Med. 1997. 186: 1347-1355.
3. Jakubowski, A., Rosa, M. D., Bixler, S., Lobb, R., and Burkly, L. C. Vascular cell adhesion molecule (VCAM)-Ig fusion protein defines distinct affinity states of the Very Late Antigen-4 (VLA-4) receptor. Cell Adhes. Commun. 1995.3: 131-142.
4. Zhang, Z., Vuori, K., Wang, H-G., Reed, J. C., and Ruoslahti, E. Integrin activation by R-ras. Cell 1996. 85: 61-69.
5. D'Souza-Schorey, C., Boettner, B., and van Aelst, L. Rac regulates integrin-mediated spreading and increased adhesion of T lymphocytes. Mol. Cell. Biol. 1998. 18: 3936-3946.
6. Van Kooyk, Y., and Figdor, C. G. Avidity regulation of integrins: the driving force in leukocyte adhesion. Curr. Opin. Cell Biol. 2000. 12: 542-547.
7. Krissansen, G. W. Integrins: Signalling and disease. The Encyclopedia of Life Sciences. MacMillan Press, 2000.
8. Hughes, P. E., and Pfaff, M. Integrin affinity modulation. Trends Cell Biol. 1998. 8: 359-364.
9. Carman, C. V., and Springer, T. A. Integrin avidity regulation: are changes in affinity and conformation underemphasized? Curr. Opin. Cell Biol. 2003. 15: 547-556.
10. Krissansen, G. W. Integrins: signalling and disease. The Encyclopedia of Life Sciences. MacMillan Press, 1999.
11. Hughes P E, Pfaff M. Integrin affinity modulation. Trends Cell Biol. 1998. 8: 359-364.
12. Sampath, R., Gallagher, P. J., Pavalko, F. M. Cytoskeletal interactions with the leukocyte integrin beta2 cytoplasmic tail. Activation-dependent regulation of associations with talin and alpha-actinin. J. Biol. Chem. 1998. 273: 33588-33594.
13. Tadokoro, S., Shattil, S. J., Eto, K., Tai, V., Liddington, R. C., de Pereda, J. M., Ginsberg, M. H., and Calderwood, D. A. Talin binding to integrin β tails: A final common step in integrin activation. Science 2003. 302: 103-106.
14. Kim, M., Carman, C. V., Springer, T. A. Bidirectional transmembrane signaling by cytoplasmic domain separation in integrins. Science 2003. 301: 1720-1725.
15. Liu, S., Calderwood, D. A., Ginsberg, M. H. Integrin cytoplasmic domain-binding proteins. J. Cell Sci. 2000 113: 3563-3571.
16. Geiger, C., Nagel, W., Boehm, T., van Kooyk, Y., Figdor, C. G., Kremmer, E., Hogg, N., Zeitlmann, L., Dierks, H., Weber, K. S., Kolanus, W. Cytohesin-1 regulates beta-2 integrin-mediated adhesion through both ARF-GEF function and interaction with LFA-1. EMBO J. 2000.19: 2525-2536.
17. Denti, S., Sirri, A., Cheli, A., Rogge, L., Innamorati, G., Putignano, S., Fabbri, M., Pardi, R., and Bianchi, E. RanBPM is a phosphoprotein that associates with the plasma membrane and interacts with the integrin LFA-1. J. Biol. Chem. 2004. 279: 13027-13034.
18. Lehnert, K., Print, C. G., Yang, Y., & Krissansen, G. W. Mucosal addressin cell adhesion molecule-1 (MAdCAM-1) costimulates T cell proliferation exclusively through integrin α4β7 (LPAM-1), whereas VCAM-1 and CS-1 peptide use α4β1 (VLA-4): Evidence for “remote” costimulation and induction of hyperresponsiveness to B7 molecules. Eur. J. Immunol. 1998. 28: 3605-3615.
19. Diamond B A et al. (1981); The New England Journal of Medicine, 1344.
20. Winter, G. and Milstein, C. (1991); Nature, 349, 293.
21. “Monocolonal Antibody Production Techniques and Applications”, Marcel Dekker, Inc: New York, 1987.
22. Holliger P, Prospero T, and Winter G, “Diabodies: small bivalent and bispecific antibody fragments” Proc Natl Acad Sci USA, 90, 6444-6448, (1993).
23. Tomlinson I and Holliger P, “Methods for generating multivalent and bispecific antibody fragments”, Methods Enzymol, 326, 461-479, (2000).
24. “Antibodies a Laboratory Manual”, Cold Spring Harbor Laboratory Press (1988).
25. Jones P T, Dear P H, Foote J, Neuberger M S, Winter G. 1986. Replacing the complementarity determining regions in a human antibody with those from a mouse. Nature 321:522-25.
26. Wang et al, J. Immunological Methods 241: 171-184, 2000.
27. Maynard, J., and Georgiou, G. 2000. Antibody engineering. Annu Rev Biomed Eng 2: 339-376.
28. M. W. Hunkapiller, R. M. Hewick, W. J. Dreyer, and L. E. Hood. 1983. High-Sequencing with a Gas-Phase Sequenator. Methods Enzymol. 91: 399.
29. Riechmann L, Clark M, Waldmann H, Winter G. 1988. Reshaping human antibodies for therapy. Nature 332:323-27.

1. An isolated peptide comprising at least the amino acid sequence of any one of:
NPxF
KSATTT
or a derivative of said peptide,
wherein x is any amino acid.

2. An isolated peptide as claimed in claim 1 wherein x is K or L.

3. An isolated peptide consisting of the amino acid sequence of any one of:
NPLFKSATTTVMNPKFAES
NPLFKSATTT
VMNPKFAES
NPLFKS
or a derivative of said peptide.

4. An isolated peptide consisting of the amino acid sequence
KALIHLSDLREYRRFEKEKLKSQWNNDNPLFKSATTTVMNPKFAES.

5. A peptide as claimed in claim 1 together with a cell membrane translocating motif.

6. A peptide as claimed in claim 5 wherein the cell membrane translocating motif is peptide-based.

7. A peptide as claimed in claim 6 wherein the cell membrane translocating motif is penetratin or a polymer of arginine.

8. An isolated nucleic acid which encodes a peptide or a derivative thereof as claimed in claim 1.

9. A nucleic acid construct comprising a nucleic acid as claimed in claim 8.

10. A construct as claimed in claim 9, wherein the construct is an expression construct.

11. An agent that comprises at least a peptide, derivative thereof, nucleic acid or construct as claimed in claim 1.

12. A pharmaceutical composition comprising a peptide or derivative thereof as claimed in claim 1 together with one or more pharmaceutically acceptable diluents, carriers and/or excipients.

13. A pharmaceutical composition comprising a nucleic acid or construct as claimed in claim 8 together with one or more pharmaceutically acceptable diluents, carriers and/or excipients.

14. A method for modulating the function of integrin β2 in a subject comprising at least the step of administering to said subject an effective amount of at least a peptide, or a derivative thereof as claimed in claim 1.

15. A method of modulating the function of integrin β2 in a subject comprising at least the step of administering to said subject an effective amount of at least a nucleic acid or construct as claimed in claim 8.

16. A a method of modulating the function of integrin β2 in an in vitro system the method comprising at least the step of administering to said system a peptide, or a derivative thereof as claimed in claim 1 or a nucleic acid, or construct as claimed in claim 8.

17. A method for the treatment of integrin β2-mediated inflammatory disorders comprising at least the step of administering to a subject in need thereof a therapeutically effective amount of at least a peptide, or a derivative thereof as claimed in claim 1.

18. A method for the treatment of integrin β2-mediated inflammatory disorders comprising at least the step of administering to a subject in need thereof a therapeutically effective amount of at least a nucleic acid or construct comprising same as claimed in claim 8.

19. The use of a peptide or a derivative thereof as claimed in any one of claim 1, or a nucleic acid or construct as claimed in claim 8, in the manufacture of a medicament for the treatment of integrin β2-mediated inflammatory disorders.

20. A method for the identification of potential β2 integrin functional interactor molecules, of the peptides as claimed in claim 1, the method comprising at least the step of bringing a potential functional interactor in contact with said peptide, or a derivative thereof, and observing whether or not binding occurs.

21. A method as claimed in claim 20 further comprising the step of determining whether or not the functional interactor influences the level of adhesion of leukocytes to β2 integrin ligands.

22. A method as claimed in claim 21 comprising the step of determining whether or not the functional interactor lowers the level of, or disrupts or prevents, adhesion of leukocytes to β2 integrin ligands.

23. The use of a peptide or derivative thereof as claimed claim 1 in identifying or screening for potential β2 integrin functional interactor molecules.

24. The use of a peptide or derivative thereof as claimed in claim 1 in designing mimetics of said peptide.

25. An antibody directed against a peptide or derivative thereof as claimed in claim 1.

26. A kit for modulating the function of integrin β2 or for the treatment of integrin β2-mediated inflammatory disorders, the kit comprising at least a peptide or derivative thereof as claimed in claim 1.

27. A kit for modulating the function of integrin β2 or for the treatment of integrin β2-mediated inflammatory disorders, the kit comprising at least a nucleic acid or construct as claimed in claim 8.
